(19) United States
(12) Patent Application Publication
(10) Pub. No.: US 2002/0176843 A1
(43) Pub. Date: Nov. 28, 2002

(54) Polyamide particles as anti-irritant agents

(75) Inventor: Isabelle Creton, Paris (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, Paris (FR)

(21) Appl. No.: 10/101,883

(22) Filed: Mar. 21, 2002

(30) Foreign Application Priority Data
Mar. 23, 2001 (FR) 01 03955

(51) Int. Cl. A61K 31/785; A61K 35/78; A61K 33/40; A61K 31/19; A61K 7/00

(52) U.S. Cl. 424/78.37; 424/616; 424/401; 424/759; 514/557; 514/725

(57) ABSTRACT

The present invention relates to the use of polyamide particles as anti-irritant agents for reducing or eliminating the irritant effect of one or more compounds with an irritant effect, preferably in a cosmetic or dermatological composition.

### BACKGROUND OF THE INVENTION

1. Field of the Invention

The present invention relates in general to the use of polyamide particles as anti-irritant agents in, for example, compositions for topical application, and for reducing the irritant effect of one or more compounds having an irritant effect. Use in dermatological and cosmetic compositions is preferred. The invention also relates to the preparation of a composition containing an irritant compound and polyamide particles.

2. Discussion of the Invention

Compositions, especially those targeted to keratin materials such as skin (such as cosmetic and dermatological compositions) often contain compounds, etc. that cause an irritant skin effect, thus diminishing their overall value.

In the context of the present invention, the irritant effect is an irritant skin effect (or skin irritation), which may be a side effect, and it is a response from the skin usually reflected by redness, pain or stinging, this response being generated by chemical compounds of natural or synthetic origin, applied topically to the skin. This irritation is often accompanied by an impairment in the epithelial function and/or structure, directly associated with the effect of the compound of irritant nature.

Disruptions induced by a compound of irritant nature are generally followed by a more or less intense response from the skin, intended to restore the homeostatic equilibrium broken, or to repair the damage caused. When the compound of irritant nature has attacked the skin, it may react with certain substances pre-existing in the cells and tissues and/or may release intracellular substances. These released substances may in turn become active on other targets in the epithelium or the dermis. This is how the cascade of reactions begins, which, by recruiting blood cells and the substances they release, give rise to the irritant process which is characterized mainly by an irritation of the skin. This process is reflected especially, to varying degrees, depending mainly on the quality and/or quantity of the applied compound and/or on the user of this compound, by dysesthetic sensations (heating, sensations of burning, itching or pruritus, sensations of stinging, tautness, etc.), by redness and/or oedema.

Compounds with an irritant effect, that is to say compounds which can cause a skin irritation, especially for individuals (users) with sensitive skin, are commonly present in cosmetic or dermatological compositions, obviously for other effects. Such compounds are termed “irritant compound” herein.

Thus, cosmetic compositions containing keratolytic active agents are used to combat ageing, and especially exfoliant active agents or active agents that promote cell renewal, such as α-hydroxy acids (in particular lactic acid, glycolic acid or citric acid), β-hydroxy acids (especially salicylic acid and 5-n-octanoylsalicylic acid) and retinoids (especially retinol and its esters). Unfortunately, if these active agents are used in excessively large amounts, they have the drawback of causing stinging, itching or tautness after they have been applied, which may lead to great discomfort. The use of these compounds, especially by users with sensitive skin, should thus be limited.

In addition, even certain compounds that are considered as inert in a cosmetic or dermatological composition, such as, for example, preserving agents, surfactants, fragrances, solvents or propellants, may have an irritant nature when they are applied to keratin materials and especially the skin, including the scalp, this irritant nature being dependant on the compound used and on the skin sensitivity of the user. Thus, it is common to use surfactants, emulsifiers or detergents in cosmetic or dermatological compositions, for example as emulsifiers in compositions in the form of water-in-oil (W/O) or oil-in-water (O/W) or triple emulsions, or, for example, as detergents in compositions for cleansing human skin. Similarly, it is common to incorporate preserving agents to ensure good conservation of the compositions, or of fragrances to give products a pleasant odour.

The compounds with an irritant nature are generally used in small doses. The use in small amount of these compounds may then prove to be of little benefit relative to the use of other compounds that are less active, but less or not at all irritant and thus used in larger amount, or relative to the final nature of the compound, such as, for example, the stability of the composition when it concerns emulsifiers or the correct conservation of the composition when it concerns preserving agents.

### OBJECT OF THE INVENTION

There is thus still a need, particularly in the cosmetic and dermatological fields, to find a means for using irritant compounds without them having an irritant nature with which the user may find fault, and thus to obtain a cosmetic or dermatological composition which, although containing compounds with an irritant side effect, is comfortable and non-irritant.

### DETAILED DESCRIPTION OF THE INVENTION

The inventor has now discovered that the use of polyamide particles makes it possible to limit or even eliminate the irritant nature of irritant compounds. This ability of polyamide particles to reduce and even eliminate the irritant nature of a composition has not been recognized. For example, WO 97/306,679 discusses moisturizing cosmetic compositions containing polyamide particles to reduce tackiness. The compositions may include preservatives. While the particles themselves are described as non-irritating, no overall effect of the particles on other components regarding reducing irritation is suggested. DE 19834819 and EP 0676195 similarly disclose compositions with polyamide particles among other additives such as preservatives, etc. but fail to note or suggest an overall anti-irritation effect of the particles. For example, the cleansing mask of EP 0676195 is suggested as comfortable and non-tightening to the skin, but this does not mean that the composition is non-irritating, or that the polyamide particles are able to reduce irritation caused by an irritating agent.

Thus, one embodiment of the present invention is the use of polyamide particles as anti-irritant agents, for example in a cosmetic composition.

The invention also relates to the use of polyamide particles to reduce the irritant effect of a composition containing one or more compounds with an irritant side effect, for example a cosmetic or dermatological composition.

The invention also relates to a process for reducing or eliminating the irritant effect of at least one compound with an irritant (side) effect present in a composition, characterized in mixing the polyamide particles and the composition, or adding the polyamide particles as a component of the composition.

The present invention especially has the advantage of eliminating or reducing the irritation that the compounds with an irritant side effect could have caused, and also of making it possible to increase the amount of compounds with an irritant side effect in, e.g., cosmetic or dermatological compositions, when compared with the amount normally used, for the purpose of improving the efficacy of the compounds. Thus, surfactants, preserving agents or keratolytic active agents such as hydroxy acids or retinoids can be used, and used in greater amounts, without any inconvenience to the user, for example.

The composition according to the invention is preferably intended for topical application, and thus preferably comprises a physiologically acceptable medium, that is to say a medium that is compatible with all keratin materials such as the skin, including the scalp, the nails, mucous membranes, eyes and hair, or any other area of human body skin.

Preferably, the polyamide particles are present in an effective amount, that is to say an amount that is sufficient for the irritant skin effect otherwise caused by the compound with an irritant (side) effect present in an irritant amount to decrease, or even disappear.

The anti-irritant effective amount of polyamide particles according to the invention is an amount of polyamide particles that is sufficient to reduce or even eliminate an irritant t keratin effect of an irritant compound. Preferably, skin is the keratin material targeted. Thus, this amount is variable as a function of the particles used, the amount and nature of the compound with an irritant nature used and/or the sensitivity of the user to this compound, and is determinable by one of ordinary skill in this art in view of this disclosure. However, by way of illustration, a composition according to the invention may generally comprise polyamide particles in a concentration ranging from 0.001% to 30% by weight, preferably from 0.01% to 20% by weight, more preferably from 0.1% to 10% by weight and better still from 0.1% to 5% by weight relative to the total weight of the composition, including all values and subranges between stated values, such as 1, 2, 3, 4, 6, 7, 8 and 9%.

Polyamide particles useful in the invention include those listed under the CTFA name of “Nylon 12” or “Nylon 6”. A mixture of particles and, for example, a mixture of Nylon-6 and Nylon-12 may be used.

The polyamide particles used in the invention include those sold under the names “Orgasol” by the company Atochem. The process for obtaining these particles is, for example, the process described in document FR-A-2 619 385 or in document EP-A-303 530. These polyamide particles are moreover known according to their various physicochemical properties under the name “polyamide 12” or “polyamide 6”.

Particles useful in the invention also include those sold under the name SP500 by the company TORAY.

According to the invention, the particles preferably have a density generally ranging from 1 g/cm³ to 1.84 g/cm³ and in particular a density ranging from 1.0 g/cm³ to 1.4 g/cm³, including all values and subranges between stated values, such as 1. 1, 1.2, 1.3, 1.5, 1.6 and 1.7 g/cm³.

The particles of the invention are preferably substantially spherical and solid; they preferably have mean sizes ranging from 5 μm to 50 μm, and more particularly ranging from 10 μm to 30 1 μm.

Compounds with an irritant (side) effect (irritant compound), which are thus liable to cause a skin irritation, especially for individuals with sensitive skin, are not limited and may especially be active agents, preserving agents, surfactants, fragrances, solvents or propellants, and mixtures thereof.

As active agents liable to have an irritant side effect, mention may be made, for example, of:

keratolytic agents such as α-hydroxy acids, for instance glycolic acid, lactic acid, malic acid, citric acid, tartaric acid and mandelic acid, and derivatives thereof; β-hydroxy acids, for instance salicylic acid and its derivatives; α-keto acids, for instance ascorbic acid or vitamin C and its derivatives; β-keto acids; retinoids, for instance retinol and its esters, retinal, retinoic acid and its derivatives (such as those described in documents FR-A-2 570 377, EP-A-199 636, EP-A-325 540 and EP-A-402 072);

anthralins (dioxyanthranol);

anthranoids (for example those described in document EP-A-319 028);

peroxides (especially benzoyl peroxide);

minoxidil and its derivatives;

lithium salts;

hair dyes or hair colorants, for instance para-phenylenediamine (p-PDA) and certain derivatives thereof such as N-phenyl-p-PDA and toluene-2,5-diamine sulphate; meta-phenylenediamine (m-PDA) and certain derivatives thereof such as toluene-3,4-diamine; ortho-phenylenediamine (o-PDA);

fragrancing alcoholic solutions (fragrances, eaux de toilette, after-shaves and deodorants);

antiperspirants (certain aluminium salts);

hair-removing active agents or permanent-waving active agents (thiols, aqueous ammonia);

depigrnenting agents (hydroquinone);

capsaicin;

anti-louse active agents (pyrethrin;

detergents (ionic and nonionic);

and mixtures thereof.

Among the salicylic acid derivatives that may be mentioned more particularly are 5-n-octanoylsalicylic acid and 5-n-dodecanoylsalicylic acid, and esters thereof.

Preserving agents that may be mentioned include phenoxyethanol, and bactericides such as octoxyglycerol or 1-(2-ethylhexyl)glyceryl ether (Sensiva SC 50 sold by the company SEPPIC), and caprylyl-glycol (or 1,2-octanediol) sold by the company Straetmans.

Surfactants which may be mentioned more particularly include anionic surfactants such as alkyl sulphates and alkyl ether sulphates, for instance lauryl sulphate and lauryl ether sulphate, and salts thereof, especially sodium salts thereof.

The composition may contain one or more irritant compounds, which may have the same activity or different activity, for example a surfactant, an active agent and a preserving agent, or a preserving agent and a surfactant.

Preferably, the amount of the irritant compound(s) in the composition of the invention is sufficient to cause a skin or other keratin material irritation in the absence of the particles (an irritant amount), the particles making it possible to attenuate or even eliminate this irritation. When referring to the invention composition, the term “irritant amount of an irritant compound” includes the sum of all irritant compounds, even where individual irritant compounds may be present in non-irritant amounts, so long as the sum is sufficient to cause a skin or other keratin material irritation in the absence of the invention particles. This amount depends on the compound(s) used and on the final nature of the composition containing it (them). It may range, for example, from 0.0001% to 50% by weight, preferably from 0.01% to 30% by weight and better still from 0.1% to 15% by weight relative to the total weight of the composition.

The physiologically acceptable medium for the compositions for topical application according to the invention may more particularly contain water and optionally a physiologically acceptable organic solvent chosen, for example, from lower alcohols containing from 1 to 8 carbon atoms and preferably 1 to 6 carbon atoms, for instance ethanol, isopropanol, propanol or butanol; polyethylene glycols containing from 6 to 80 ethylene oxides; polyols, for instance propylene glycol, isoprene glycol, butylene glycol, glycerol or sorbitol. This medium may also be an anhydrous medium, especially an oily medium containing ails and/or fatty substances other than oils.

When the physiologically acceptable medium is aqueous, it preferably has a pH that is compatible with the skin, preferably ranging from 3 to 8 and better still from 4.5 to 7.

When the composition comprises an aqueous or aqueous-alcoholic medium, it is possible to add a fatty phase (or oily phase) to this medium, so that the compositions of the invention are softer and more nourishing. The oily phase usually contains at least one oil. As oils that may be used in the composition of the invention, mention may be made, for example, of:

hydrocarbon-based oils of animal origin, such as perhydrosqualene;

hydrocarbon-based oils of plant origin, such as liquid triglycerides of fatty acids containing from 4 to 10 carbon atoms, for instance heptanoic or octanoic acid triglyceride or alternatively, for example, sunflower oil, corn oil, soybean oil, marrow oil, grape seed oil, sesame oil, hazelnut oil, apricot oil, macadamia oil, arara oil, castor oil, avocado oil, caprylic/capric acid triglycerides for instance those sold by the company Stearineries Dubois or those sold under the names Miglyol 810, 812 and 818 by the company Dynamit Nobel, jojoba oil or karite butter oil;

synthetic esters and synthetic ethers, especially of fatty acids, for instance oils of formulae R¹COOR² and R¹OR² in which R¹ represents a fatty acid residue containing from 8 to 29 carbon atoms and R² represents a branched or unbranched hydrocarbon-based chain containing from 3 to 30 carbon atoms, such as, for example, purcellin oil, isononyl isononanoate, isopropyl myristate, 2-ethylhexyl palmitate, 2-octyldodecyl stearate, 2-octyldodecyl erucate or isostearyl isostearate; hydroxylated esters such as isostearyl lactate, octyl hydroxystearate, octyldodecyl hydroxystearate, diisostearyl malate, triisocetyl citrate and fatty alkyl heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tatraisostearate;

linear or branched hydrocarbons of mineral or synthetic origin, such as volatile or non-volatile liquid paraffins, and derivatives thereof, petroleum jelly, polydecenes, and hydrogenated polyisobutene such as parlean oil;

fatty alcohols containing from 8 to 26 carbon atoms, for instance cetyl alcohol, stearyl alcohol and a mixture thereof (cetylstearyl alcohol), octyldodecanol, 2-butyloctanol, 2-hexyldecanol, 2-undecylpentadecanol, oleyl alcohol or linoleyl alcohol;

alkoylated and especially ethoxylated fatty alcohols such as oleth-12;

partially hydrocarbon-based and/or :silicone-based fluoro oils, for instance those described in document JP-A-2 295 912. Examples of fluoro oils which may also be mentioned include perfluoromethylcyclopentane and perfluoro-1,3-dimethylcyclohexane, sold under the names “Flutes PC1®” and “Flutes PC3®” by the company BNFL Fluorochemicals;

perfluoro-1,2-dimethylcyclobutane; perfluoroalkanes such as dodecafluoropentane and tetradecafluorohexane, sold under the names “PF 5050®” and “PF 5060®” by the company 3M, or alternatively bromoperfluorooctyl sold under the name “Foralkyl®” by the company Atochem; nonafluoromethoxybutane sold under the name “MSX 4518®” by the company 3M and nonafluoro ethoxyisobutane; perfluoromorpholine derivatives, such as the 4-trifluoromethylperfluoromorpholine sold under the name “PF 5052®” by the company 3M;

silicone oils, for instance volatile or non-volatile polymethylsiloxanes (PDMSs) containing a linear or cyclic silicone chain, that are liquid or pasty at room temperature, especially cyclopolydimethylsiloxanes (cyclomethicones) such as cyclohexasiloxane; polydimethylsiloxanes comprising alkyl, alkoxy or phenyl groups, that are pendent or at the end of a silicone chain, these groups containing from 2 to 24 carbon atoms; phenylsilicones, for instance phenyltrimethicones, phenyldimethicones, phenyltri-methylsiloxydiphenylsiloxanes, diphenyldimethicones, diphenylmethyldiphenyltrisiloxanes, 2-phenylethyl-trimethylsiloxysilisates and polymethylphenylsiloxanes;

mixtures thereof.

In the list of oils mentioned above, the expression “hydrocarbon-based oil” means any oil mainly comprising carbon and hydrogen atoms, and optionally ester, ether, fluoro, carboxylic acid and/or alcohol groups.

The other fatty substances that may be present in the oily phase are, for example, fatty acids containing from 8 to 30 carbon atoms, for instance stearic acid, lauric acid, palmitic acid and oleic acid; waxes, for instance lanolin, beeswax, carnauba wax or candelilla wax, paraffin wax, lignite wax or microcrystalline waxes, ceresin or ozokerite, synthetic waxes such as polyethylene waxes, Fischer-Tropsch waxes; gums such as silicone gums (dimethiconol); silicone resins such as trifluoromethyl-C₁₋₄-alkyl-dimethicone and trifluoropropyldimethicone; and silicone elastomers, for instance the products sold under the names “KSG” by the company Shin-Etsu, under the names “Trefil”, “BY29” or “EPSX” by the company Dow Coming or under the names “Gransil” by the company Grant Industries.

These fatty substances may be chosen in a varied manner by a person skilled in the art so as to prepare a composition having the desired properties, for example in terms of consistency or texture.

The compositions according to the invention may be in any form, such as forms conventionally used for topical application, and especially in the form of aqueous, aqueous-alcoholic or oily solutions, oil-in-water (O/W) or water-in-oil (W/O) or multiple emulsions, aqueous or oily gels, liquid, pasty or solid anhydrous products, or dispersions of a fatty phase in an aqueous phase with the aid of spherules, these spherules possibly being polymer nanoparticles such as nanospheres and nanocapsules, or lipid vesicles of ionic and/or nonionic type. These compositions may be prepared according to the usual methods.

In addition, the compounds used according to the invention may be more or less fluid and may have the appearance of a white or coloured cream, an ointment, a milk, a lotion, a serum, a. paste or a mousse. They may optionally be applied to the skin in the form of an aerosol. They may also be in solid form, and for example in the form of a stick.

According to one particular embodiment of the invention, the composition according to the invention is a water-in-oil (W/O) or oil-in-water (O/W) emulsion. The proportion of the oily phase in the emulsion may range from 5% to 80% by weight and preferably from 5% to 50% by weight relative to the total weight of the composition. The oils, emulsifiers and co-emulsifiers used in the composition in emulsion form may be chosen from those conventionally used in cosmetics or dermatology. The emulsifier and the co-emulsifier are generally present in the composition in a proportion ranging from 0.3% to 30% by weight and preferably from 0.5% to 20% by weight relative to the total weight of the composition. The emulsion may also contain lipid vesicles.

The emulsions generally contain at least one emulsifier chosen from amphoteric, anionic, cationic and nonionic emulsifiers, used alone or as a mixture. The emulsifiers are chosen in a suitable manner depending on the emulsion to be obtained (W/0 or O/W emulsion).

As emulsifying surfactants that may be used for the preparation of the W/O emulsions, mention may be made, for example, of alkyl esters or ethers of sorbitan, of glycerol or of sugars; silicone surfactants, for instance dimethicone copolyols such as the mixture of cyclomethicone and of dimethicone copolyol sold under the name “DC 5225 C” by the company Dow Coming, and alkyldimethicone copolyols, such as the laurylmethicone copolyol sold under the name “Dow Corning 5200 Formulation Aid” by the company Dow Corning and the cetyl dimethicone copolyol sold under the name Abil EM 90® by the company Goldschmidt.

For the O/W emulsions, examples of emulsifiers which may be mentioned include nonionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alkyl ethers; sugar esters, for instance sucrose stearate.

According to one particular embodiment of the invention, the polyamide particles used according to the invention are combined with one or more calmant active agents chosen from the other anti-irritant agents and/or anti-inflammatories, these calmants reinforcing the anti-irritant effect of the polyamide particles. Thus, a subject of the invention is also a composition for topical application, containing, in a physiologically acceptable medium, polyamide particles and at least one calmant.

Calmants which may be mentioned, for example, include:

allantoin;

β-glycyrrhetinic acid, extracts containing it such as, for example, extract of Glycyrrhiza glabra (liquorice) and complexes containing it, such as the allantoin/glycyrrhetinic acid complex;

lyophilized or non-lyophilized plankaons, extracts thereof and complexes thereof; i a

escin and plant extracts containing it, for instance extract of common horse-chestnut;

xanthine derivatives, for instance diethylaminoethyl-theophylline hydrochloride;

waters and extracts (for example aqueous, aqueous-alcoholic or aqueous-glycolic extracts) of flowers and plants, for instance cornflower water, camomile water, mint water, lime water, rose water, extracts of peony, extracts of hawthorn, extracts of yarrow, extracts of mallow, extracts of marigold, extracts of sweet clover, extracts of sage, elder water, extracts of ginkgo biloba, extracts of arnica, extracts of oregano, extracts of green tea, extracts of waterlily flowers, extracts of iris and extracts of birch bark;

asiatic acid and plant extracts containing it, for instance Centella asiatica;

bisabolol;

fruit extracts, for instance extract: of pineapple or extract of papaya;

algae, especially of the type such as Laminaria (for example red or brown algae);

gamma-orizanol;

pyrrolidone carboxylates and especially of zinc (Zn-PCA) or of copper (Cu-PCA);

oils of plant origin, for instance canola seed oil and karite oil;

essential oils, for example of coriander, of balm, of lavender, of mint and of camomile, and mixtures thereof;

acexamic acid and transexamic acid (trans-4-aminomethylcyclohexanecarboxylic acid);

ursolic acid and extracts containing it, for example extract of rosemary leaf,

polysaccharides containing fucose, for instance Fucogel 1000, sold by Solabia (aqueous solution comprising 1% polysaccharide solids comprising fucose, galactose and galacturonic acid);

electrolytes and in particular an aqueous mixture comprising from 30% to 35% magnesium chloride, from 20% to 28% potassium chloride, from 3° s to 10% sodium I chloride, from 0.2% to to calcium chloride, from 0.1% to 0.6% magnesium bromide and from 0.1% to 0.5% of insolubles, the said mixture being referred to herein as “Dead Sea bath salts” since it corresponds to the main salts contained in the Dead Sea;

galactolipids obtained, for example, from oat, such as, for example, digalactosyl diglyceride or monogalactosyl diglyceride;

amino acids, derivatives thereof and salts thereof, such as the sodium salt of amino acids grafted onto cocoyl chains, sold in the form of a mixture under the name Sepicalm S by the company SEPPIC, capryloylglycine sold under the name Lipacide C8G by the company SEPPIC and the mixture of ca:pryloylglycine, cinnamon and sarcosine sold under the name Sepicontrol A5 by the company SEPPIC;

TNF-alpha antagonists such as lisophylline, A802715, sulfasalazine, CDP-571 (anti-TNF-alpha antibody) and MDL-201112;

substance P antagonists such as sencLide, spantide II, the peptides described in document EP-A-680 749, and the extracts of filamentous bacteria described in document EP-A-761 204;

and mixtures thereof.

The amount of calmant(s) may range, for example, from 0.001% to 20% by weight and preferably from 0.01% to 15% by weight relative to the total weight of the composition.

The compositions of the invention may also contain adjuvants that are common in cosmetics or dermatology, such as hydrophilic or lipophilic gelling agents, hydrophilic or lipophilic active agents other than those mentioned above, preserving agents, antioxidants, solvents, fragrances, fillers, lipophilic or hydrophilic sunscreeens, bactericides, odour absorbers, dyestuffs, salts and polymers (for example acrylate/Dimethicone copolymer sold under the name KP-561 by Shin-Etsu, as dispersant). The amounts of these various adjuvants are those conventionally used in the field under consideration, and, for example, from 0.01% to 20% relative to the total weight of the composition. Depending on their nature, these adjuvants may be introduced into the fatty phase, into the aqueous phase and/or into lipid spherules.

As fillers which may be used in the composition of the invention, mention may be made, for example, besides pigments, of silica powder; talc; polyethylene powders; microspheres based on acrylic copolymers, such as those based on ethylene glycol dimethacrylate/lauryl methacrylate copolymer sold by the company Dow Corning under the name Polytrap; expanded powders such as hollow microspheres and especially the microspheres sold under the name Expancel by the company Kemanord Plast or under the name Micropearl F 80 ED by the company Matsumoto; powders of natural organic materials such as crosslinked or noncrosslinked corn starch, wheat starch or rice starch, such as the powders of starch crosslinked with octenyl succinate anhydride, sold under the name Dry-Flo by the company National Starch; silicone resin microbeads such as those sold under the name Tospearl by the company Toshiba Silicone; and mixtures thereof. These fillers may be present in amounts ranging from 0% to 20% by weight and preferably from 1% to 10% by weight relative to the total weight of the composition.

The composition used according to the invention may contain one or more UV screening agents (or sunscreens) which may be a chemical screening agent or a physical sunblock or a mixture of such screening agents.

As chemical sunscreens that may be used in the composition of the invention, the composition of the invention may comprise any UVA and UVB screening agents that may be used in cosmetics.

UVB screening agents that may be mentioned, for example, include:

(1) salicylic acid derivatives, in particular homomenthyl salicylate and octyl salicylate;

(2) cinnamic acid derivatives, in particular 2-ethylhexyl p-methoxycinnamate, sold by the company Givaudan under the name Parsol MCX;

(3) liquid β,β′-diphenylacrylate derivatives, in particular 2-ethylhexyl α-cyano-α:,β′-diphenylacrylate or octocrylene, sold by the company BASF under the name Uvinul N539;

(4) p-aminobenzoic acid derivatives;

(5) 4-methylbenzylidene camphor sold by the company Merck under the name Eusolex 6300;

(6) 2-phenylbenzimidazole-5-sulphonic acid sold under the name Eusolex 232 by the company Merck;

(7) 1,3,5-triazine derivatives, in particular:

2,4,6-tris [p-(2′-ethylhexyl-1′-oxycarbonyl)anilino]1,3,5-triazine sold by the company BASF under the name Uvinul T150, and

dioctylbutamidotriazone sold by the company Sigma 3V under the name UV Asorb HEB;

(8) mixtures of these screening agents.

UVA screening agents that may be mentioned, for example, include:

(1) dibenzoylmethane derivatives, in particular 4-(tert-butyl)-4′-methoxydibenzoylmethane sold by the company Givaudan under the name Parsol 1789;

(2) benzene-1,4-bis(3-methylidene-10-camphorsulphonic acid) optionally in partially or totally neutralized form, sold under the name Mexoryl SX by the company Chimex;

(3) benzophenone derivatives, for example:

2,4-dihydroxybenzophenone (benzophenone-1),

2,2′,4,4′-tetrahydroxybenzophenone (benzophenone-2),

2-hydroxy-4-methoxybenzophenone (benzophenone-3), sold under the name Uvinul M40 by the company BASF,

2-hydroxy-4-methoxybenzophenone-5-sulphonic acid (benzophenone-4) and also its sulphonate form (benzophenone-5), sold by the company BASF under the name Uvinul MS40, 2,2′-dihydroxy-4,4′-dimethoxybenzophenone (benzophenone-6), 5-chloro-2-hydroxybenzophenone (benzophenone-7), 2,2′-dihydroxy-4-methoxybenzophenone (benzophenone-8);

the disodium salt of 2,2′-dihydroxy-4,4′-dimethoxy-benzophenone-5,5′-disulphonic diacid (benzophenone-9),

2-hydroxy-4-methoxy-4′-methylbenzophenone (benzophenone-10),

benzophenone-11,

2-hydroxy-4-(octyloxy)benzophenone (benzophenone-12);

(4) silane or polyorganosiloxane derivatives containing a benzophenone group;

(5) anthranilates, in particular menthyl anthranilate sold by the company Haarman & Reimer under the name Neo Helipan MA;

(6) compounds comprising at least two benzazolyl groups or at least one benzodiazolyl group per molecule, in particular 1,4-bis(benzimidazolyl)phenylene-3,3′,5,5′-tetrasulphonic acid and its salts, sold by the company Haarman and Reimer;

(7) silicon derivatives of N-substituted benzimidazolylbenzazoles or of benzofurylbenzazoles, and in particular:

2-[1-[3-[1, 3, 3, 3-tetramethyl-1-[(trimethylsilyl) oxy]-disiloxanyl]propyl]-1H-benzimidazol-2-yl]benzoxazole,

2-[1-[3-[1, 3, 3, 3-tetramethyl-1-[(trimethylsilyl) oxy]-disiloxanyl]propyl]-1H-benzimidazol-2-yl]benzo-thiazole,

2-[1-(3-trimethylsilanylpropyl)-1H-benzimidazol-2-yl]benzoxazole,

6-methoxy-1,1 ′-bis-(3-trimethylsilanylpropyl)-1H,1′H-2,2′]bibenzimidazolylbenzoxazole,

2-[1-(3-trimethylsilanylpropyl)-1H-benzimidazol-2-yl]-benzothiazole, which are described in patent application EP-A-1 028 120;

(8) triazine derivatives, and in particular 2,4-bis {[4-(2-ethylhexyloxy)-2-hydroxy]phenyl-6-(4-methoxyphenyl)-1,3,5-triazine sold by the company Ciba Geigy under the name Tinosorb S, and 2,2′-methylenebis[6-(2H-benzotriazol-2-yl)-4-(1,1,3,3-tetramethylbutyl)phenol] sold by the company Ciba Geigy under the name Tinosorb M; and

(9) mixtures thereof.

A mixture of several of these screening agents and a mixture of UVB screening agents and UVA screening agents may also be used, as may mixtures with physical sunblock screening agents.

Physical sunblock screening agents which may be used include titanium oxide (titanium dioxide in amorphous form or crystallized in rutile and/or anatase form), zinc oxide, iron oxide, zirconium oxide or cerium oxide, or mixtures thereof. These metal oxides may be in the form of particles of micrometric or nanometric size (nanopigments). In the form of nanopigments, the mean particle sizes :range, for example, from 5 to 100 nm. Nanopigments are preferably used in the composition of the invention.

The composition according to the invention containing polyamide particles and a calmant is suitable, depending on the other compounds contained therein, in particular for body and hair hygiene and especially for treating acne-prone skin, for regrowth of the hair, for preventing hair loss, for combating the greasy appearance of the skin or the hair, protecting against the harmful aspects of sunlight or in the treatment of physiologically dry skin, for preventing and/or combating light-induced or chronological ageing, and for making up the skin, the eyelashes and mucous membranes (the lips). Thus, when it contains anti-ageing active agents, it may be intended for combating ageing of the shin, and in particular for combating wrinkles and/or fine lines in the skin, and for giving a smooth complexion. When it contains surfactants and especially detergent surfactants, it may be intended for cleansing and/or removing make-up from the skin, the hair or any other keratin material.

The composition of the invention may thus constitute a care or treatment composition for the skin (including the scalp), keratin fibres (hair, eyelashes or eyebrows), the nails or the lips, or an antisun or artificial tanning composition, or alternatively a product for cleansing or removing make-up from the skin, the hair, the eyebrows or the eyelashes, a deodorant product or a fragrancing compound. It is then generally uncoloured or faintly coloured, and it may optionally contain cosmetic or dermatological active agents. It may then be used as a care base for the skin or the lips (lip balms, for protecting the lips against cold and/or sunlight and/or wind), as a day or night care cream for facial skin and/or body skin. It may also be in the form of a medicated or unmedicated, colouring or non-colouring shampoo, and of a conditioning compound.

The composition according to the invention may also constitute a coloured cosmetic composition and especially a make-up composition for the skin, keratin fibres (hair or eyelashes) and/or mucous membranes, in particular a foundation, a blusher, a face powder, an eyeshadow, a mascara, an eyeliner, a concealer compound in stick form, a nail varnish, a lipstick or a lip gloss, optionally having care or treatment properties, or a body tattoo.

The example which follows serves to illustrate the invention without, however, being limiting in nature. The names are, depending on the case, the chemical names or CTFA names (International Cosmetic Ingredient Dictionary and Handbook) and the amounts are given as percentage by weight, except where otherwise mentioned.

### EXAMPLE: O/W Emulsion

| Phase A1 | | |
|---|---|---|
| Glycerol | | 7% |
| EDTA (sodium salt) | | 0.05% |
| Phenoxyethanol/parabens (preserving agents) | | 1% |
| Water | qs | 100% |
| Phase A2 | | |
| Salicylic acid | | 2% |
| Triethanolamine | | 2.05% |
| Water | | 7% |
| Phase B1 | | |
| Cetearyl alcohol | | 1.2% |
| Oleth-12 | | 0.3% |
| Stearyl alcohol | | 1% |
| Glyceryl stearate/PEG 100 stearate | | 2.5% |
| (Arlacel 165) | | |
| Hydrogenated polyisobutene | | 3% |
| Octyl methoxycinnamate (Parsol MCX) | | 5% |
| Acrylate/dimethicone copolymer (KP-551) | | 0.6% |
| (dispersant) | | |
| Phase B2 | | |
| Fragrance | | 0.5% |
| Cyclopentasiloxane | | 7% |
| Octoxyglycerol (Sensiva SC50) | | 0.5% |
| Phase C | | |
| Sepigel 305 (SEPPIC) | | 0.7% |
| Phase D | | |
| Nylon 12 (Orgasol 2002 Extra D Nat Cos sold by Atochem) | | 8% |
| Phase E | | |
| Modified starch (Dry Flo) | | 3% |

Procedure: phases A1 and A2, preheated to 80° C., are mixed together and phase B1, preheated to 80° C., is introduced into the mixture with stirring. A fluid emulsion is obtained, which is cooled to 60° C., followed by introducing phase B2 and then phase D and, after cooling to 40° C., phase E, with stirring.

A non-irritant white cream capable of treating greasy skin is obtained.

French patent application 0103955 filed Mar. 23, 2001, is incorporated herein by reference, as are all publications, documents, standards, references and texts mentioned herein.

As noted above, the polyamide particles of the invention can be used in a process of making non-irritating compositions. The process comprises providing an anti-irritant effective amount of the invention particles and the irritant compound(s) in the same composition in any manner of mixing, contacting, etc., and in any order of addition (“mixing”). Included for example is a method for reducing the irritation effect caused by an irritant compound comprising providing an anti-irritant effective amount of polyamide particles in the composition.

Also included in the invention is an article of manufacture comprising the invention polyamide particles and, associated therewith in the form of instructions, packaging, literature, etc., indicia in the form of writing or otherwise indicating the use of the particles in rendering irritant compounds non-irritating, particularly in cosmetic and dermatological compositions.

1. A non-irritating composition comprising an anti-irritant effective amount of polyamide particles and an irritant amount of an irritant compound.

2. The composition of claim 1, comprising 0.001% to 30% by weight polyamide particles relative to the total weight of the composition.

3. The composition of claim 1, wherein the polyamide particles have a density ranging from 1 g/cm³ to 1.84 g/cm³.

4. The composition of claim 1, wherein the polyamide particles have a mean size ranging from 5 μm to 50 μm.

5. The composition according to claim 1, wherein said polyamide particles are selected from the group consisting of Nylon-6, Nylon-12, and mixtures thereof.

6. The composition of claim 1, wherein the polyamide particles are present in an amount ranging from 0.01% to 20% by weight relative to the total weight of the composition.

7. The composition according to claim 1, wherein said irritant compound is selected from the group consisting of active agents, preserving agents, surfactants, fragrances, solvents, propellants, and mixtures thereof.

8. The composition of claim 1, wherein the irritant compound is selected from the group consisting of α-hydroxy acids, β-hydroxy acids, α-keto acids, β-keto acids, retinoids, anthralins, anthranoids, peroxides, minoxidil and its derivatives, lithium salts, hair dyes, hair colorants, fragrancing alcoholic solutions, antiperspirants, hair-removing active agents, permanent-waving active agents, depigmenting agents, capsaicin, anti-louse active agents, detergents, bactericides, anionic surfactants, and mixtures thereof.

9. The composition of claim 1, wherein the irritant compound is selected from the group consisting of glycolic acid, lactic acid, malic acid, citric acid, tartaric acid, mandelic acid and derivatives thereof, salicylic acid and its derivatives, 5-n-octanoylsalicylic acid, esters thereof, 5-n-dodecanoylsalicylic acid, esters thereof, retinol and its esters, retinal, retinoic acid and its derivatives, phenoxyethanol, octoxyglycerol, caprylyl glycol, alkyl sulphates, salts thereof, alkyl ether sulphates, salts thereof, and mixtures thereof.

10. The composition according to claim 1, wherein the amount of irritant compound present ranges from 0.0001% to 50% by weight relative to the total weight of the composition.

11. The composition of claim 1, further comprising a physiologically acceptable medium.

12. The composition of claim 1, wherein said composition is a cosmetic or dermatological composition.

13. A process for rendering a composition comprising an irritant amount of an irritant compound non-irritating, comprising mixing said composition with an anti-irritant effective amount of polyamide particles.

14. The process of claim 13, wherein said composition is a cosmetic or dermatological composition comprising a physiologically acceptable medium.

15. A cosmetic process for treating keratin material, comprising applying the composition of claim 1 to keratin material.

16. The method of claim 16, wherein the keratin material is human skin.

17. A method for reducing the irritation caused by an irritant compound in a composition comprising providing an anti-irritant effective amount of polyamide particles in said composition.
